(19) United States
(12) Patent Application Publication
(10) Pub. No.: US 2001/0044142 A1
(43) Pub. Date: Nov. 22, 2001

(54) Microbial reductase useful for the stereoselective reduction of a racemic tetralone

(76) Inventors: Maria S. Brown, Pawcatuck, CT (US); Ronald W. Fedechko, Mystic, CT (US); John W. Wong, East Lyme, CT (US)

Correspondence Address: Gregg C. Benson, Pfizer Inc., Patent Department, MS 4159, Eastern Point Road, Groton, CT, 06340, US

(21) Appl. No.: 09/834,098

(22) Filed: Apr. 12, 2001

Related U.S. Application Data
(60) Provisional application No. 60/200,413, filed on Apr. 28, 2000.

(51) Int. Cl. C12N 9/04; C12P 7/22

(52) U.S. Cl. 435/156; 435/190

(57) ABSTRACT

The present invention relates to novel compositions of matter comprising an enzyme activity capable of carrying out the following stereoselective reduction of a racemic tetralone:

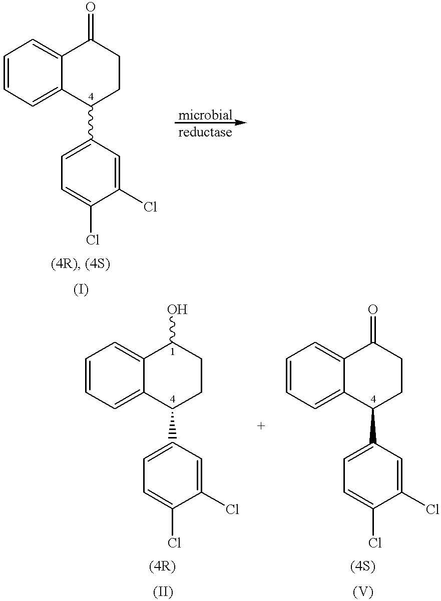

The chiral tetralone can be used in the synthesis of sertraline, well known to be useful, for example, as an antidepressant and anorectic agent, and in the treatment of chemical dependencies, anxiety-related disorders, premature ejaculation, cancer and post-myocardial infarction.

### CROSSREFERENCE TO RELATED APPLICATION

This application claims priority from U.S. Provisional Patent Application No. 60/200,413 filed Apr. 28, 2000, the benefit of which is hereby claimed under 37 C.F.R. §1.78(a)(3).

### FIELD OF THE INVENTION

The present invention relates to a novel composition of matter comprising an enzyme activity found to be present in Hansenula polymorpha ATCC No. 26012. The enzyme activity comprises a reductase which is capable of stereoselectively reducing racemic 4-(3,4-dichlorophenyl)-3,4-dihydro-1 (2H)-naphthalenone (here-inafter also referred to as “racemic tetralone”) to the (4S) enantiomer of 4-(3,4-dichlorophenyl)-3,4-dihydro-1 (2H)-naphthalenone (hereinafter also referred to as “chiral tetralone” or “(4S) tetralone”). The present invention further relates to the use of the novel composition of matter to prepare chiral tetralone from racemic tetralone. The chiral tetralone produced by the reduction of the invention can be further reacted to prepare sertraline which is known to be useful, for example, as an antidepressant.

### BACKGROUND OF THE INVENTION

It has been reported that Hansenula polymorpha ATCC No. 26012, also deposited as ATCC No. 74449, contains an enzyme activity capable of stereoselectively reducing racemic tetralone to chiral tetralone. Copending and commonly assigned U.S. patent application Ser. No. 09/427,424 (the '424 application).

The chiral tetralone prepared by the processes of the '424 application may be further reacted to prepare pure cis-(1S)(4S)-N-methyl-4-(3,4-dichlorophenyl)-1,2,3,4-tetrahydro-1-naphthaleneamine, commonly referred to as sertraline. Sertraline is well known to be useful, for example, as an antidepressant and anorectic agent, and in the treatment of chemical dependencies, anxiety-related disorders, premature ejaculation, cancer and post-myocardial infarction.

A composition of matter comprising an enzyme activity capable of stereoselectively reducing racemic tetralone to chiral tetralone is also provided by the present invention. A particular aspect of the processes disclosed in the '424 application is also now provided.

All of the documents cited herein, including the foregoing, are incorporated by reference herein in their entireties.

### SUMMARY OF THE INVENTION

This invention provides a composition of matter comprising an enzyme activity, where said enzyme activity is capable of stereoselectively reducing racemic tetralone to chiral tetralone.

The compositions of matter comprising the enzyme activity of this invention can be provided by (1) preparing a crude extract of induced cells of Hansenula polymorpha ATCC No. 26012, Hansenula polymorpha ATCC No. 74449, or any suitable mutant thereof, or (2) preparing a crude extract of induced cells of Hansenula polymorpha ATCC No. 26012, Hansenula polymorpha ATCC No. 74449, or any suitable mutant thereof, removing the deoxyribonucleic acid (DNA) therefrom, salting out the polypeptides therefrom, and concentrating (dialyzing) the resultant resuspended polypeptide pellet.

The preparations of either (1) or (2) may then be optionally (and preferably) followed by column chromatography to further isolate the subject microbial reductase activity, e.g., using any suitable affinity column(s), anion exchange column(s), size exclusion column, and the like, as well as any suitable combination thereof, in any suitable order (with desalting, as needed), as will be appreciated by those skilled in the art based on the present description and the attendant claims. In a preferred embodiment, the preparation of (2) is followed by column chromatography using a suitable affinity column.

A preferred affinity column is a Blue Sepharose CL-6B column commercially available from Pharmacia Biotech.

Preferred anion exchange columns include, for example, a POROS20 HQ column, a POROSHP2 column with a 1M NaCl gradient (gradient to OM NaCl), a POROS20QE column with a 1 M NaCl gradient, and a POROS DEAE Sepharose column with a 1 M NaCl, all commercially available from PerSeptive Biosystems, Inc.; and a DEAE Sepharose Fast Flow column commercially available from Pharmacia Biotech.

A preferred size exclusion column is a Superdex 200HR 10/30 column commercially available from Pharmacia Biotech.

In a particularly preferred embodiment, a composition of matter comprising the enzyme activity of this invention and the fractions in which it is present according to this invention are as follows:

(a) present in a fraction from a crude preparation of Hansenula polymorpha ATCC No. 26012, Hansenula polymorpha ATCC No. 74449, or any other suitable mutant thereof, where said crude fraction is prepared by: inducing cells of Hansenula polymorpha ATCC No. 26012, Hansenula polymorpha ATCC Hansenula polymorpha ATCC No. 74449, or any other suitable mutant thereof, with an amount of the compound of Formula (I) under suitable conditions to permit the induction of the enzyme activity capable of stereoselectively reducing racemic tetralone to chiral tetralone, centrifuging said induced cells, resuspending said centrifuged cells in a breaking buffer comprising beads, rupturing said resuspended cells under suitable conditions to permit disruption of said cells and retention of an appreciable amount of said enzyme activity, centrifuging said breaking buffer after said rupturing, retaining the supernatant of said centrifuged breaking buffer, and adding a protein stabilizing agent to said supernatant;

(b) present in a fraction of the fraction described in (a) above, where said fraction is obtained by: adding a DNA precipitating agent to an amount of said fraction described in (a) above, centrifuging said crude extract, retaining the supernatant of said centrifuged crude extract, adding a protein precipitating agent to said supernatant of said centrifuged crude extract to achieve about 48% fractional saturation, centrifuging said supernatant having about 48% fractional saturation, retaining said supernatant and adding a protein precipitating agent to said supernatant to achieve about 75% fractional saturation, centrifuging said supernatant having about 75% fractional saturation, retaining the pellet resulting from said centrifugation, resuspending the proteins comprising said pellet in a buffer and desalting and then concentrating said proteins in said buffer;

(c) present in a fraction of the fraction described in (b) above, where said fraction is obtained by: loading an amount of said fraction described in (b) above onto a column comprising a material capable of reversibly associating with said proteins comprising said fraction described in (b) and having said enzyme activity, eluting said reversibly associated proteins from said column using an NADPH gradient, assaying each eluted fraction for said enzyme activity, and pooling said eluted fractions having said enzyme activity;

(d) present in a fraction of the fraction described in (c) above, where said fraction is obtained by: desalting an amount of said fraction described in (c) above, loading an amount of said desalted fraction onto a column comprising an anion exchange material and capable of reversibly binding said proteins of said desalted fraction and having said enzyme activity, eluting said reversibly associated proteins from said column using a salt gradient, assaying each eluted fraction for said enzyme activity, and pooling said eluted fractions having said enzyme activity;

(e) present in a fraction of the fraction described in (d) above, where said fraction is obtained by: desalting an amount of said fraction described in (d) above, loading an amount of said fraction described in (d) above onto a column comprising a weak anion exchange material and capable of reversibly binding said proteins of said desalted fraction and having said enzyme activity, eluting said reversibly associated proteins from said column using a salt gradient, assaying each eluted fraction for said enzyme activity, and pooling said eluted fractions having said enzyme activity; and

(f) present in a fraction of the fraction described in (e) above, where said fraction is obtained by: desalting an amount of said fraction described in (e) above, concentrating said desalted fraction, loading an amount of said concentrated fraction onto a column comprising a size exclusion material, and eluting a fraction comprising a polypeptide of from about 110,000 D to about 200,000 D and having said enzyme activity;

where said activity is present in said fractions when an amount of racemic tetralone is stereoselectively reduced to an amount of chiral tetralone.

The present invention also provides processes for carrying out the following stereospecific reduction:

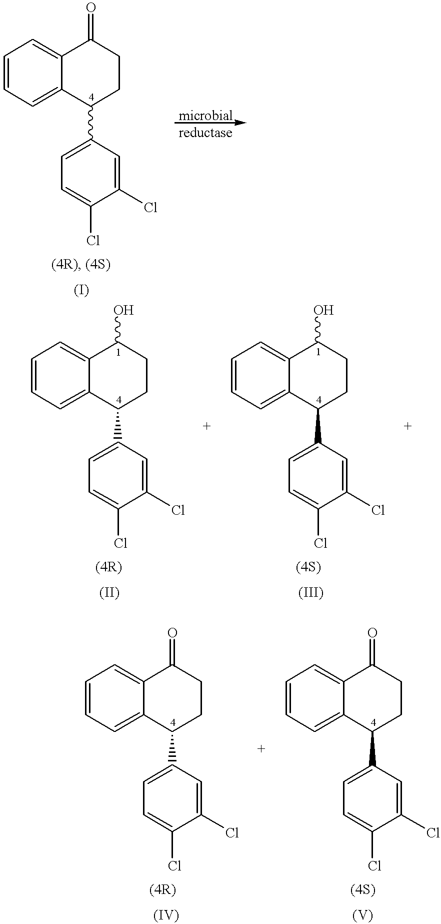

which comprises: contacting a compound of Formula (I) with a composition of matter comprising an enzyme activity capable of accomplishing the subject reduction and a co-factor for said enzyme, and incubating the resulting mixture under conditions sufficient to yield more of the compound of Formula (II) than the compound of Formula (III), thus leaving more of the compound of Formula (V) unreacted than the compound of Formula (IV) unreacted;

where said composition of matter comprising said enzyme activity has the following characteristics:

(a) present in a fraction from a crude preparation of Hansenula polymorpha ATCC No. 26012, Hansenula polymorpha ATCC No. 74449, or any other suitable mutant thereof, where said crude fraction is prepared by: inducing cells of Hansenula polymorpha ATCC No. 26012, Hansenula polymorpha ATCC Hansenula polymorpha ATCC No. 74449, or any other suitable mutant thereof, with an amount of the compound of Formula (I) under suitable conditions to permit the induction of the enzyme activity capable of stereoselectively reducing racemic tetralone to chiral tetralone, centrifuging said induced cells, resuspending said centrifuged cells in a breaking buffer comprising beads, rupturing said resuspended cells under suitable conditions to permit disruption of said cells and retention of an appreciable amount of said enzyme activity, centrifuging said breaking buffer after said rupturing, retaining the supernatant of said centrifuged breaking buffer, and adding a protein stabilizing agent to said supernatant;

(a) present in a fraction of the fraction described in (a) above, where said fraction is obtained by: adding a DNA precipitating agent to an amount of said fraction described in (a) above, centrifuging said crude extract, retaining the supernatant of said centrifuged crude extract, adding a protein precipitating agent to said supernatant of said centrifuged crude extract to achieve about 48% fractional saturation, centrifuging said supernatant having about 48% fractional saturation, retaining said supernatant and adding a protein precipitating agent to said supernatant to achieve about 75% fractional saturation, centrifuging said supernatant having about 75% fractional saturation, retaining the pellet resulting from said centrifugation, resuspending the proteins comprising said pellet in a buffer and desalting and then concentrating said proteins in said buffer;

(b) present in a fraction of the fraction described in (b) above, where said fraction is obtained by: loading an amount of said fraction described in (b) above onto a column comprising a material capable of reversibly associating with said proteins comprising said fraction described in (b) and having said enzyme activity, eluting said reversibly associated proteins from said column using an NADPH gradient, assaying each eluted fraction for said enzyme activity, and pooling said eluted fractions having said enzyme activity;

(c) present in a fraction of the fraction described in (c) above, where said fraction is obtained by: desalting an amount of said fraction described in (c) above, loading an amount of said desalted fraction onto a column comprising an anion exchange material and capable of reversibly binding said proteins of said desalted fraction and having said enzyme activity, eluting said reversibly associated proteins from said column using a salt gradient, assaying each eluted fraction for said enzyme activity, and pooling said eluted fractions having said enzyme activity;

(d) present in a fraction of the fraction described in (d) above, where said fraction is obtained by: desalting an amount of said fraction described in (d) above, loading an amount of said fraction described in (d) above onto a column comprising a weak anion exchange material and capable of reversibly binding said proteins of said desalted fraction and having said enzyme activity, eluting said reversibly associated proteins from said column using a salt gradient, assaying each eluted fraction for said enzyme activity, and pooling said eluted fractions having said enzyme activity; and

(e) present in a fraction of the fraction described in (e) above, where said fraction is obtained by: desalting an amount of said fraction described in (e) above, concentrating said desalted fraction, loading an amount of said concentrated fraction onto a column comprising a size exclusion material, and eluting a fraction comprising a polypeptide of from about 110,000 D to about 200,000 D and having said enzyme activity.

The subject stereospecific reduction may also be represented by:

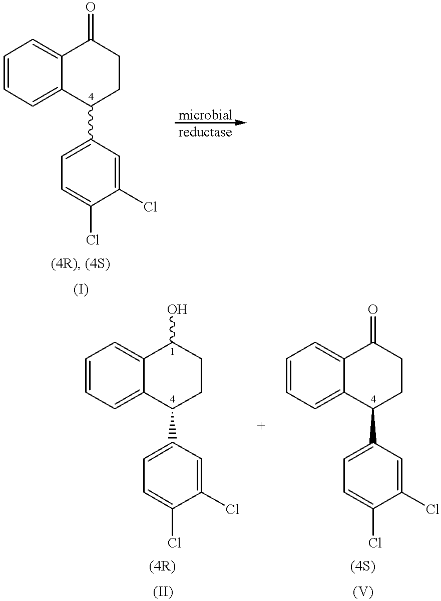

which comprises: contacting a compound of Formula (I) with a composition of matter comprising an enzyme activity capable of accomplishing the subject reduction and a co-factor for said enzyme, and incubating the resulting mixture under conditions sufficient to yield the (4R) tetralol of Formula (II) and to leave substantially unreacted the (4S) tetralone of Formula (V); where said enzyme activity has the characteristics provided hereinabove.

The stereoselective reduction further optionally comprises the separation of the (4S) tetralone of Formula (V) from the (4R) tetralol of Formula (II). The (4R) tetralol may then be oxidized to produce the (4R) tetralone, which is then reacted, e.g., with a base, to produce racemic tetralone of Formula (I) and the subject stereoselective reduction may be repeated to result in even more of the desired (4S) tetralone of Formula (V), i.e., the (4S) enantiomer of the racemic tetralone of Formula (I).

The present invention provides processes which comprise the stereoselective reduction of a compound of Formula (I) to a compound of Formula (II) by: contacting a compound of Formula (I) with a composition of matter comprising an enzyme activity capable of accomplishing the subject reduction and a co-factor for said enzyme, and incubating the resulting mixture under conditions sufficient to yield a compound of Formula (II), whereby substantially more of the compound of Formula (V) remains unreacted than the compound of Formula (IV) and substantially more of the compound of Formula (II) is produced than the compound of Formula (III).

Hansenula polymorpha ATCC No. 26012, also deposited as ATCC No. 74449, or any suitable mutant thereof, appreciably reduces only one enantiomer of the compound of Formula (I), to give the corresponding alcohol, i.e., the compound of Formula (II), while leaving the other enantiomer of the compound of Formula (I), i.e., the compound of Formula (V), substantially unreacted.

As discussed earlier, the processes of the present invention further optionally include the separation, e.g., carried out using crystallization or chromatography, of the compound of Formula (V) from the compounds of Formulae (II)-(IV), and the use of such separated compound of Formula (V) in the synthesis of sertraline using any known methods therefor.

As also discussed earlier, it is preferred to oxidize the isolated (4R) tetralol of Formula (II) to the (4R) tetralone of Formula (IV). It is then further preferred to racemize, preferably by reacting the (4R) tetralone with a base, the (4R) tetralone of Formula (IV) to the racemic tetralone of Formula (I). The oxidation and racemization recycles the unwanted (4R) tetralol for another round of stereoselective microbial reduction according to the processes of the present invention. The recycling of the unwanted (4R) tetralol increases the amount of the desired (4S) tetralone and decreases the amount of unwanted (4R) tetralol discarded. The oxidation and the racemization of the oxidized product may be carried out using any suitable known methods therefor.

### DETAILED DESCRIPTION OF THE INVENTION

Those skilled in the art will fully understand the terms used herein to describe the present invention; nonetheless, the following terms or abbreviations used herein, are as described immediately below. “°C” means degrees-Centigrade; “cm” means centimeter or centimeters; “co-factor” means any suitable co-factor for the subject enzyme activity such as, for example, NADH (nicotinamide adenine dinucleotide), NADPH (nicotinamide adenine dinucleotide phosphate), FADH (flavin adenine dinucleotide), FMNH (flavin mononucleotide), and/or PQQ (2,7,9-tricarboxy-1H-pyrrolo [2,3-f] quinoline-4,5-dione), or any suitable co-factor which occurs with the enzyme in the microorganism; “C.V.” means column volume; “D” means daltons; “DTT” means dithiothreitol; “EDTA” means ethylene-diaminetetraacetic acid; “g” means gram or grams; “h” means hour or hours; “HPLC” means high performance liquid chromatography; “L” means liter or liters; “M” means molar (concentration); “mg” means milligram or milligrams; “microbial reductase” means the subject enzyme activity capable of accomplishing the subject reduction; “min” means minute or minutes; “mL” means milliliter or milliliters; “mm” means millimeter or millimeters; “%” means percent; “rpm” means revolutions per minute; and “sec” means second or seconds.

The usefulness of the novel compositions of matter of this invention is illustrated, for example, by the processes provided by the present invention which comprise the stereoselective reduction of a compound of the Formula (I) to a compound of the Formula (II):

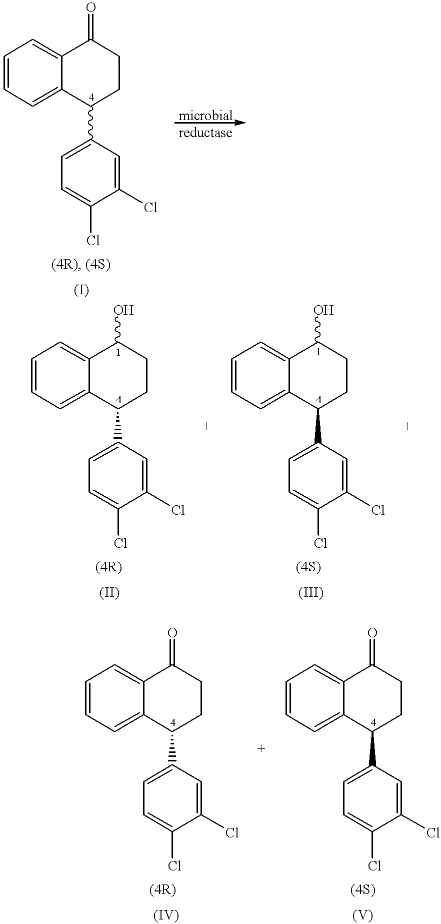

by contacting a compound of Formula (I) with a composition of matter comprising an enzyme activity capable of accomplishing the subject reduction and a co-factor for said enzyme, and incubating the resulting mixture under conditions sufficient to yield a compound of Formula (II), whereby substantially more of the compound of Formula (V) remains unreduced than the compound of Formula (IV) and substantially more of the compound of Formula (II) is produced than the compound of Formula (III);

where said composition of matter comprising said enzyme activity has the following characteristics:

(a) present in a fraction from a crude preparation of Hansenula polymorpha ATCC No. 26012, Hansenula polymorpha ATCC No. 74449, or any other suitable mutant thereof, where said crude fraction is prepared by: inducing cells of Hansenula polymorpha ATCC No. 26012, Hansenula polymorpha ATCC Hansenula polymorpha ATCC No. 74449, or any other suitable mutant thereof, with an amount of the compound of Formula (I) under suitable conditions to permit the induction of the enzyme activity capable of stereoselectively reducing racemic tetralone to chiral tetralone, centrifuging said induced cells, resuspending said centrifuged cells in a breaking buffer comprising beads, rupturing said resuspended cells under suitable conditions to permit disruption of said cells and retention of an appreciable amount of said enzyme activity, centrifuging said breaking buffer after said rupturing, retaining the supernatant of said centrifuged breaking buffer, and adding a protein stabilizing agent to said supernatant;

(b) present in a fraction of the fraction described in (a) above, where said fraction is obtained by: adding a DNA precipitating agent to an amount of said fraction described in (a) above, centrifuging said crude extract, retaining the supernatant of said centrifuged crude extract, adding a protein precipitating agent to said supernatant of said centrifuged crude extract to achieve about 48% fractional saturation, centrifuging said supernatant having about 48% fractional saturation, retaining said supernatant and adding a protein precipitating agent to said supernatant to achieve about 75% fractional saturation, centrifuging said supernatant having about 75% fractional saturation, retaining the pellet resulting from said centrifugation, resuspending the proteins comprising said pellet in a buffer and desalting and then concentrating said proteins in said buffer;

(c) present in a fraction of the fraction described in (b) above, where said fraction is obtained by: loading an amount of said fraction described in (b) above onto a column comprising a material capable of reversibly associating with said proteins comprising said fraction described in (b) and having said enzyme activity, eluting said reversibly associated proteins from said column using an NADPH gradient, assaying each eluted fraction for said enzyme activity, and pooling said eluted fractions having said enzyme activity;

(d) present in a fraction of the fraction described in (c) above, where said fraction is obtained by: desalting an amount of said fraction described in (c) above, loading an amount of said desalted fraction onto a column comprising an anion exchange material and capable of reversibly binding said proteins of said desalted fraction and having said enzyme activity, eluting said reversibly associated proteins from said column using a salt gradient, assaying each eluted fraction for said enzyme activity, and pooling said eluted fractions having said enzyme activity;

(e) present in a fraction of the fraction described in (d) above, where said fraction is obtained by: desalting an amount of said fraction described in (d) above, loading an amount of said fraction described in (d) above onto a column comprising a weak anion exchange material and capable of reversibly binding said proteins of said desalted fraction and having said enzyme activity, eluting said reversibly associated proteins from said column using a salt gradient, assaying each eluted fraction for said enzyme activity, and pooling said eluted fractions having said enzyme activity; and

(f) present in a fraction of the fraction described in (e) above, where said fraction is obtained by: desalting an amount of said fraction described in (e) above, concentrating said desalted fraction, loading an amount of said concentrated fraction onto a column comprising a size exclusion material, and eluting a fraction comprising a polypeptide of from about 110,000 D to about 200,000 D and having said enzyme activity.

As would be understood by those skilled in the art, the compound of Formula (I), racemic tetralone, is a mixture of (4S) tetralone and (4R) tetralone as shown below:

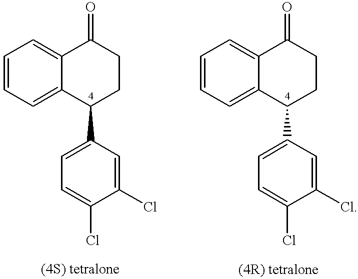

The compounds, or more specifically, the tetralols of Formula (II) are:

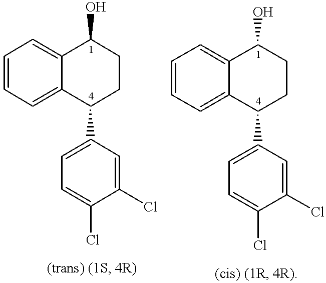

The compounds, or more specifically, the tetralols of Formula (III) are:

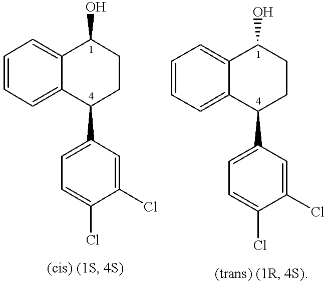

The compounds of Formulae (II) and (III) are disclosed and claimed in the aforementioned U.S. Pat. No. 5,750,794.

The desired compound of Formula (V) may be isolated as described below from the undesired compounds of Formula (II), and any of the compounds of Formulae (III) or (IV) which may have been either produced or remained unreacted, respectively, depending upon, e.g., the microorganism selected and the conditions of incubation.

The compounds of Formula (II) may be converted to a compound of Formula (I), e.g., by oxidization and racemization, and run through the subject stereoselective reduction to result in yet another amount of the (4S) tetralone of Formula (V).

It will be understood by those skilled in the art based on the present description that suitable microorganisms from which a crude extract comprising the subject enzyme activity may be derived include: Hansenula polymorpha ATCC No. 26012 and Hansenula polymorpha ATCC No. 74449; and mutants thereof which are known or otherwise obtainable by those skilled in the relevant art and able, despite such mutation, to accomplish the stereoselective reduction disclosed herein (“suitable mutants”). Preferred microorganisms will be those having an enzyme activity which substantially reduces the (4R) tetralone while leaving the (4S) tetralone substantially unreacted.

A lyophilized sample of Hansenula polymorpha ATCC No. 26012 (originally contributed by D. W. Levine) was deposited with the ATCC located at 10801 University Boulevard, Manassas, Va., 20110-2209, U.S.A., under the terms of the Budapest Treaty on Jun. 26, 1998. This newly deposited culture was given the new deposit number of ATCC No. 74449.Hence, it is also preferred in the present invention that the microorganism is Hansenula polymorpha ATCC No. 74449. All restrictions on the availability to the public of the microorganism culture so deposited will be irrevocably removed upon the issuance of a patent from the specification of the present invention.

The microorganisms from which the crude extract will be may be prepared by any suitable method known to those skilled in the relevant art. An example of a suitable method for the preparation of a microorganism from a commercially purchased stock is provided below. The method provided below may be used for any microorganism suitable for use in preparing the subject novel composition of matter comprising the subject enzyme activity for use in the present inventive process, and those skilled in the art would understand from the description provided herein how to modify any part of any of the procedures, e.g., method of growing or preparing the microorganism, method of preparing the crude extract derived therefrom, method of purifying the subject enzyme activity, method of contacting of the racemic tetralone with the microorganism to induce the production of the subject enzyme activity, method of contacting of the racemic tetralone with the novel composition of matter comprising the subject enzyme activity, growth medium components and conditions, e.g., temperature, pH and the like; or incubation conditions; to achieve the desired result in any particular process, procedure, or protocol.

Cultures of Hansenula polymorpha ATCC No. 26012 can be obtained from the ATCC, and an example of a suitable method for its preparation from such a commercially purchased stock is provided immediately below. A culture so obtained is added to a suitable growth medium, and is incubated with shaking until growth occurs, both steps as would be appreciated by those skilled in the art. The culture, thus prepared, can be used to inoculate slants, with portions of these slants frozen as master stocks. Alternatively, liquid stock cultures can be prepared to which glycerol is added to from about 10% to about 20% which are then frozen at about −80° C., preferably in small cryotubes.

As would be understood by those skilled in the art for any microorganism selected, and as provided specifically hereinafter in the example for Hansenula polymorpha ATCC No. 26012 (or ATCC No. 74449), a suitable method for preparing the microorganism is as follows: the microorganism is inoculated from a frozen stock culture such as described above (about a 17% glycerol stock) into a flask or a glass tube with a metal closure containing a growth medium (containing an aliquot from a sterile solution which includes Tween® 80, glycerol and distilled water) whose composition is described in more detail below. The fermentation is carried out at temperatures ranging from about 22° C. to about 32° C., and preferably at about 29° C., with suitable shaking, preferably from about 200 rpm to about 220 rpm, and most preferably, at about 210 rpm. Where so desired, the pH of the growth medium can be maintained by the use of suitable buffers incorporated into the fermentation medium and/or periodically adjusted by addition of either base or acid as so required.

Any suitable duration of growth of the microorganism, contacting of the microorganism with the compound of Formula (I) to induce the production of the subject enzyme activity (using, e.g., a 2 mM stock solution prepared by adding about 1.16 mL of an about 5 mg/mL solution of the compound of Formula (I) (in ethanol) to about 8.84 mL ethanol, and preferably brought to room temperature and mixed to re-dissolve prior to use), and incubation of the compound of Formula (I) with the microorganism may be used in the present invention. Suitable growth of the microorganism may be achieved, e.g., within about 24 hours, at which time a suitable aliquot of a solution of racemic tetralone in a suitable solvent, preferably ethanol, may be added to the culture. The fermentation may then be continued for, e.g., from about two to about six days, and preferably, e.g., for about five days, at which time the fermentation broth may be extracted using any suitable extraction method whereby a suitable solvent, such as, for example, ethyl acetate, methyl isobutylketone, methyl ethylketone, methylene chloride, and the like, preferably, ethyl acetate, removes the organic components from the fermentation broth. After extraction of the fermentation broth and separation of the organic and aqueous phases, the compounds comprising the organic residue may be determined using any suitable method, such as, for example, chromatography, preferably, chiral HPLC.

Any suitable growth medium may be used in the process of the present invention, and the suitable growth medium will contain a source or sources of assimilable carbon, assimilable nitrogen and inorganic salts containing essential minerals. In general, many carbohydrates such as, for example, glucose, maltose, mannose, sucrose, starch, glycerin, millet jelly, molasses, soy bean, and the like, can be used as sources of assimilable carbon. Sources of assimilable nitrogen include, for example, materials such as yeast and casein hydrolysates, primary yeast, yeast extracts, cottonseed flour, soybean solids, wheat germ, meat extracts, peptone, corn steep liquor, and ammonium salts. Suitable inorganic salt nutrients for use in the culture medium of the present invention include, for example, the customary salts containing sodium, iron, magnesium, potassium, cobalt, phosphate, and the like. More particularly, growth media suitable for use in the present invention include, for example:

(a) dextrose (about 20 g), yeast extract (about 5 g), soy flour (about 5 g), NaCl (about 5 g), K₂HPO₄ (about 5 g), and distilled H₂O (about 1 L), pH adjusted to about pH 7.0 with H₂SO_{4(aq.)};

(b) dextrin (about 10 g), beef extract (about 3 g), ardamine pH (about 5 g), NZ amine type E (about 5 g), MgSO₄ 7H₂O (about 0.5 g), KH₂PO₄ (about 0.37 g), CaCO₃ (about 0.5 g), and distilled H₂O (about 1 L), pH adjusted to about pH 7.1 with HCl _{(aq.)}, followed by a second stage of glucose (about 10 g), Hy-Case SF® (about 2 g), beef extract (about 1 g), corn steep liquor (about 3 g), and distilled H₂O (about 1 L), pH adjusted to about pH 7.0;

(c) glucose (about 10 g), corn steep liquor (about 6 g), KH₂PO₄ (about 3 g), CaCO₃ (about 3.5 g), soybean oil (crude, about 2.2 mL), yeast extract (about 2.5 g), and distilled H₂O (about 1 L), pH adjusted to from about pH 7.0 to about pH 7.3 with HCl _{(aq.)};

(d) malt syrup (about 20 g), soybean meal (about 5 g), casein (about 1 g), dried yeast (about 1 g), NaCl (about 5 g), and distilled H₂O (about 1 L);

(e) lactose (about 75 g), Pharmamedia® (substitute yeast extract, about 40 g), CaCO₃ (about 10 g), Na₂SO₄ (about 4 g), and distilled H₂O (about 1 L);

(f) ISP #2 (see, e.g., page 460 of the Handbook of Microbial Media by R. M. Atlas, edited by L. C. Parks, CRC Press, Inc., 1993, (“Handbook”));

(g) ISP #3 (see, page 460 of the Handbook);

(h) ISP#4 (see, page 461 of the Handbook);

(i) ISP#5 (see, pages 461-462 of the Handbook); and the like.

A particularly preferred growth medium is 2× of (a) provided above.

Reference to particular buffers, media, reagents, contacting or culture conditions, and the like, is not intended to be limiting, but should be read to include all such related materials that those of ordinary skill in the art would recognize as being of interest or value in the particular context in which the discussion herein is presented. For example, it is often possible to substitute one buffer system or culture medium for another, such that a different but known way is used to achieve the same goals as those to which the use of a suggested method, material or composition is directed. Moreover, it should be understood that the present invention includes the scaling-up of the subject process for commercial purposes.

Hence, as would be understood by those of ordinary skill in the art, variation of the growth medium, the conditions of fermentation, and/or the amount of racemic tetralone may be altered to control the yield of the resultant compounds and their relative rates of production. In general, the techniques employed in the present invention will be chosen with regard to industrial efficiency. The growth media, conditions of fermentation and relative amounts of microorganism, and racemic tetralone described herein are merely illustrative of the wide variety of media, fermentation conditions and amounts of starting materials which may be suitably employed in the present invention as would be appreciated by those skilled in the art, and are not intended to be limiting in any way.

Any suitable method of contacting the compound of Formula (I) with the microorganism, or with a crude extract, or with any other fraction or pooled fractions, may be used in the present invention. The compound of Formula (I) may be contacted with the material comprising the subject enzyme activity in any suitable order. For example, where inducing the microorganism to produce the subject enzyme activity, the compound of Formula (I) may be added to a medium, such as a culture broth, comprising the microorganism, free or immobilized, or some combination thereof; or the medium may comprise the compound of Formula (I) and the microorganism may then be added to such medium; or the compound of Formula (I) and the microorganism may be added together to such medium. Likewise, where assaying for the subject enzyme activity in a crude extract, or with any other fraction or pooled fractions, the substrate, the compound of Formula (I), may be contacted in any suitable manner with the extract or fraction(s). Those skilled in the art will understand from the description provided herein how to modify any part of the subject process as so desired.

The processes of the present invention are readily carried out. Thus, the microorganism is fermented in the presence of the racemic tetralone, represented by Formula (I), to induce the production of the desired enzyme activity, the enzyme activity which is capable of accomplishing the stereoselective reduction of racemic tetralone to chiral tetralone. The crude extract is prepared as described hereinbelow. The further fractionation of the crude extract is carried out as also described hereinbelow. The crude extract, fractions derived from subsequent fractionations, and the resultant fraction as described in (f) above, are then assayed for the desired reductase activity, e.g., to obtain the fraction of most interest, namely, the one or pooled fractions comprising the subject enzyme activity. The resultant fraction (e.g., free, immobilized, etc.) having the desired activity, together with a suitable co-factor, may then be used to modify racemic tetralone, and more particularly, to reduce the undesired (4R) enantiomer of the racemic ketone to its corresponding alcohol, represented by Formula (II), while leaving the desired (4S) enantiomer, represented by Formula (V), substantially unreacted, thereby, in one step, resulting in the optically enriched (4S) enantiomer. The (4S) enantiomer may then be further reacted by methods well known to those skilled in the relevant art such as described, for example, in U.S. Pat. Nos. 4,536,518; 4,777,288; 4,839,104; 4,855,500; 4,940,731; 4,962,128; 5,082,970; 5,130,338; 5,196,607; 5,248,699; 5,442,116; 5,463,126; 5,466,880; 5,597,826; and 5,750,794; and, in the aforementioned paper of W. M. Welch, Jr. et al., to ultimately yield sertraline. The activity, methods for testing activities, dosages, dosage forms, methods of administration and background information concerning sertraline are set forth, for example, in U.S. Pat. Nos. 4,536,518; 4,777,288; and 4,839,104; and the aforementioned paper by W. M. Welch, Jr. et al.

The crude preparation or extract of Hansenula polymorpha ATCC No. 26012, Hansenula polymorpha ATCC No. 74449, or any other suitable mutant thereof, may be prepared in any suitable manner such that the subject enzyme activity is substantially maintained. A preferred method of preparation is provided in the Examples section of the present description provided hereinbelow.

As described in (a) above, after the cells are induced to produce the subject enzyme activity, the cells are spun or centrifuged to generate a cell pellet and a supernatant comprising undesired material, e.g., any residual amount of the compound of Formula (I) that may be present as the case may be. Then, the cells are resuspended in a suitable volume of breaking buffer, where the breaking buffer preferentially comprises beads to assist with the rupturing or shearing of the cells. Preferred beads include, for example, 0.5 mm diameter beads obtained from Biospec (Biospec Products, Bartlesville, Okla. 74005) and used, e.g., with a Biospec Bead-Beater, as will be discussed in more detail in the Examples provided hereinbelow. The rupturing is allowed to occur for a suitable period of time and then the breaking buffer is spun or centrifuged, the supernatant retained, and a protein stabilizing agent added thereto. Preferred breaking buffers include, for example, a buffer comprising 0.1 M KH₂PO₄, pH 7.0 (about 54.44 g into about 3.9 L dH₂O, adjust to about pH 7.0 with about 20% KOH, final volume to 4 L), about 15% glycerol (measure volume and add about 85% glycerol to a final concentration of about 15%), about 3 mM DTT (added immediately before use), Turkey Eggwhite Trypsin Inhibitor (about 1 mg/mL), about 2 mM EDTA. Preferred protein stabilizing agents include, for example, glycerol.

The crude extract may then be further fractionated where so desired (as described above in (b)) by precipitating out any deoxyribonucleic acid (DNA) present in the fraction. This may be accomplished by any suitable method such as, for example: adding a suitable amount of a DNA precipitating agent to an amount of the crude extract and, after a suitable period of time, spinning or centrifuging this solution and retaining the supernatant, adding a protein precipitating agent to the supernatant to achieve about 48% fractional saturation, repeating the centrifugation, retaining the resultant supernatant therefrom and adding an additional amount of said protein precipitating agent to achieve about 75% fractional saturation, centrifuging once again but, in this case, retaining the pellet and discarding the supernatant, resuspending the pelleted material in a suitable buffer, then desalting and concentrating the resultant polypeptides in the buffer. Preferred DNA precipitating agents include, for example, about 10% polyethylenimine. Preferred protein precipitating agents include, for example, ammonium sulfate. A preferred resuspension buffer comprises 10 mM KH₂PO₄, pH 7.0, 3 mM DTT (added immediately before use), 2 mM EDTA, and 15% glycerol. A preferred method of desalting is by using an Amicon ultrafiltration cell concentrator with a YM10 membrane (Amicon, Inc. Beverly, Mass. 01915).

An amount of the polypeptide-containing fraction obtained as described in the immediately preceding paragraph may be further fractionated where so desired using any suitable affinity column, a column having an affinity for dehydrogenases. The polypeptide(s) of interest, those having the subject enzyme activity (reductase activity), reversibly associate with (e.g., bind to) the column material and may then be eluted with from the column using, e.g., an NADPH gradient. Preferred affinity columns include, for example, a Blue Sepharose CL-6B column commercially available from Pharmacia Biotech (Uppsala, Sweden) and having the following characteristics with respect to its preferred use in this invention: 10 g of powder is packed into 2.6 cm×about 9 cm column (packing buffer comprising about 10 mM KH₂PO₄, about pH 7.0, about 3 mM DTT (added immediately before use), and about 2 mM EDTA), mean bead size is about 90 microns (from about 45 to about 165 microns), packed at about 10 mUmin offline, run at about 5 mL/min, 0 to about 3 mM NADPH gradient in binding buffer (comprising 10 mM KH₂PO₄, pH 7.0, 3 mM DTT (added immediately before use), 2 mM EDTA, and 15% glycerol) over 10 column volumes (about 114.68 min total run time counting a 2 column volume equilibration at 0 NADPH), where the enzyme activity of interest has eluted (elution buffer comprising 10 mM KH₂PO₄, pH 7.0, 3 mM DTT (added immediately before use), 2 mM EDTA, 15% glycerol, and 5 mM NADPH) in from about 0.8 to about 3 mM NADPH. The fractions eluted from the column may be assayed for the subject enzyme activity and then pooled, if so desired, prior to the next (optional yet preferred) fractionation step.

An amount of the fraction(s) eluted from the affinity column as discussed in the immediately preceding paragraph may then be further fractionated where so desired after desalting the fraction(s) using any suitable column comprising anion exchange material and capable of reversibly associating with (e.g., binding to) the subject enzyme activity. The enzyme activity may then be eluted from the anion exchange column using a salt gradient, with each fraction being preferably assayed for the subject enzyme activity, and those fractions comprising the subject enzyme activity pooled where so desired. Preferred anion exchange columns include, for example, a POROS20HQ column commercially available from PerSeptive Biosystems, Inc. (Framingham, Mass. 01701) and having the following characteristics with respect to its preferred use in this invention: 4.6×100 mm (column volume=about 1.662 mL), particle size is about 20 microns, run at about 5 mL/min, gradient of binding buffer (comprising about 10 mM KH₂PO₄, about pH 7.0, about 3 mM DTT (added immediately before use), about 2 mM EDTA, about 15% glycerol and about 1 M NaCl (about pH 8)(100:0 to 75:25)) over 40 column volumes (about 14.96 min total run time), and having a 5 column volume equilibration at starting conditions (a 40 C.V. gradient), where the enzyme activity of interest has eluted at from about 0.08 M to about 0.24 M NaCl. The fractions eluted from the anion exchange column may be assayed for the subject enzyme activity and then pooled, if so desired, prior to the next (optional yet preferred) fractionation step. Additional preferred anion exchange columns include a POROSHP2 column with a 1M NaCl gradient, a POROS20QE column with a 1M NaCl gradient, and a POROS DEAE Sepharose column with a 1M NaCl, all commercially available from PerSeptive Biosystems, Inc.

An amount of the fraction(s) eluted from the anion exchange column and having the subject enzyme activity as discussed in the immediately preceding paragraph may be further fractionated where so desired after desalting the fraction(s) using any suitable column comprising anion exchange material and capable of reversibly associating with (e.g., binding to) the subject enzyme activity. The enzyme activity may then be eluted from the anion exchange column using a salt gradient, with each fraction being preferably assayed for the subject enzyme activity, and those fractions comprising the subject enzyme activity pooled where so desired. Preferred anion exchange columns include, for example, a DEAE (diethylaminoethyl) Sepharose Fast Flow column commercially available from Pharmacia Biotech and having the following characteristics with respect to its preferred use in this invention: 4.6×100 mm (C.V.=about 1.662 mL), particle size is about 90 microns (from about 45 to about 165 microns), packed at about 2 mL/min with binding buffer (comprising about 10 mM KH₂PO₄, about pH 7.0, about 3 mM DTT (added immediately before use), and about 2 mM EDTA), gradient of binding buffer (comprising about 10 mM KH₂PO₄, about pH 7.0, about 3 mM DTT (added immediately before use), about 2 mM EDTA, about 15% glycerol and about 1 M NaCl (about pH 7)(100:0 to 50:50)) over 40 C.V. at about 1 m/min (about 84.76 min total run time), and having a 5 column volume equilibration at starting conditions (a 40 C.V. gradient, a 2 C.V. gradient to 0:100%, then a 4 C.V. “wash” of 0:100), where the enzyme activity of interest has eluted at from about 0.27 M to about 0.33 M NaCl. The fractions eluted from the anion exchange column may be assayed for the subject enzyme activity and then pooled, if so desired, prior to the next (optional yet preferred) fractionation step.

An amount of the fraction(s) eluted from the anion exchange column and having the subject enzyme activity as discussed in the immediately preceding paragraph may be further fractionated, where so desired, after desalting the fraction(s)(e.g., as described above), using any suitable column comprising a size exclusion material capable of allowing the separable elution of a fraction(s) comprising the subject enzyme activity, a fraction(s) comprising a polypeptide having a molecular weight of from about 110,000 D to about 200,000 D and preferably is comprised of a size exclusion material allowing for the elution of a fraction(s) comprising a polypeptide of from about 150,000 D to about 160,000 D. Preferred size exclusion columns include, for example, a Superdex 200HR 10/30 column commercially available from Pharmacia Biotech and having the following characteristics with respect to its preferred use in this invention: 10×30 cm, bead diameter of from about 13 microns to about 15 microns, a flow rate of about 1 m/min of a buffer comprising about 10 mM KH₂PO₄, about 3 mM DTT, about 2 mM EDTA, about 0.15 M NaCl, about pH 6.8 for about 1.5 column volumes, with a total run time of about 35.43 min, where the enzyme activity of interest has eluted at in fractions 11 through 13, with fraction 11 having the majority of the subject enzyme activity, calibrated to about 158,000 D molecular weight. As those skilled in the art will appreciate, the fraction(s) may be assessed for the subject enzyme activity as discussed above. An amount of the eluted fraction(s), pooled or not as the case may be, may be subjected to SDS-PAGE to further resolve the polypeptide of interest. Preferred PAGE methods include, for example, the NOVEX NuPAGE Electrophoresis System which is commercially available from Novex (San Diego, Calif. 92121) and includes an instruction booklet. This system is further described in more detail hereinbelow in the Examples section. The amount of the subject enzyme activity may then be compared with the band(s) resolved by the use of the gel, and the appropriate band excised for amino acid sequencing, and other conventional molecular biology techniques to determine the sequence of DNA encoding the polypeptide. When the band on the gel that migrated with a substantial amount of the subject enzyme activity was subjected to limited acetic acid digestion (using about 10% acetic acid) and partial internal (blocked amino terminus) amino acid sequences were determined and then compared with a database containing amino acid sequences, the BLAST searches show some homology to a yeast dehydrogenase with about a 174,000 D molecular weight (accession number: Z48179).

The amount of protein in the crude extract as well as in the fractions derived from the crude extract may be determined using any suitable method of measuring protein. Preferred methods include, for example, an assay commercially available from BioRad (BioRad Total Protein Assay, Bio-Rad Laboratories, Hercules, Calif. 94547), as will be discussed in more detail in the Examples provided hereinbelow.

The presence, as well as the amount, of the subject enzyme activity may be assessed for each of the crude extract and the fractions derived therefrom, using any, part, or all of the column chromatography procedures provided hereinabove, by determining, for example, whether an enzyme activity is present in the given extract or fraction by contacting the extract or fraction with an amount of the compound of Formula (I), the substrate, in the presence of a suitable co-factor, and ascertaining whether the racemic tetralone is stereoselectively reduced to chiral tetralone. The structure of the compound(s) produced by such a reduction can be specifically determined through the use of a suitable HPLC column such as, for example, a Chiracel OD HPLC column (detects the alcohol products as well as the two tetralones) or a Chiracel OK column (detects the two tetralones, the (4S) and the (4R)), both commercially available from Waters (34 Maple Street, Milford, Mass. 01757). A preferred Chiracel OD HPLC column (Waters) has the following characteristics with respect to its preferred use in this invention: 4.6×250 mm+50 mm guard, a flow rate of about 0.9 mL/min, a total run time of about 45 min with a 5 microliter injection, a mobile phase of hexane:isopropyl alcohol (95:5), with detection at about 210 nanometers.

Any suitable methods for isolating and/or purifying any of the products of the subject process may be used in the present invention including filtration, extraction, crystallization, column chromatography, thin-layer chromatography, preparative low pressure liquid chromatography or HPLC, or any suitable combination of such methods.

Further, one of skill in the art would appreciate that the unwanted corresponding alcohol of the (4R) tetralone, the compound of formula (II), produced by the processes disclosed herein, may be recycled, e.g., oxidized and racemized as discussed earlier herein, by any suitable known method to a racemic tetralone of formula (I), and the processes of the present invention repeated to result in, once again, the desired (4S) tetralone of formula (V). The oxidation of the (4R) tetralol to the (4R) ketone can be done by methods known to those skilled in the art. The racemization reaction may be performed in any suitable manner but is generally performed at a temperature of from about 0° C. to about 10° C., preferably from about 25° C. to about 65° C. The (4R) tetralone is reacted with a base at a temperature of from about 25° C. to about 85° C., preferably from about 50° C. to about 65° C. Suitable bases for this racemization reaction include potassium t-butoxide, sodium hydroxide, sodium methoxide and potassium hydroxide. A preferred base is potassium t-butoxide.

### EXAMPLES

As discussed hereinabove, the processes of the present invention are readily carried out. The specific examples provided hereinbelow illustrate preferred methods for fermenting the microorganism in the presence of the substrate (i.e., the compound of Formula (I)), for preparing the crude extract of the induced microorganism, for preparing each of the fractions discussed hereinabove, and for assaying the subject enzyme activity (e.g., for deciding which fractions to keep, pool, etc., as the case may be).

These examples also show that the crude extract can be further fractionated to provide a composition of matter comprising an enzyme activity capable of accomplishing the subject reduction and being substantially free of unwanted material. The enzyme activity of the further fractionated or substantially purified composition of matter is substantially increased over that of the crude extract and, those skilled in the art will appreciate that using a substantially purified composition of matter comprising the desired enzyme activity or microbial reductase has advantages over using a crude extract, e.g., less unwanted material that may cause degradation, e.g., of the substrate (i.e. the compound of Formula (I), a dimunition of the desired enzyme activity, etc.

The reduction, as performed by the crude extract or any fraction or pooled fraction(s) as the case may be, in one step, results in the optically enriched (4S) enantiomer or chiral tetralone. The (4S) enantiomer may then be further reacted by methods well known to those skilled in the art to ultimately yield sertraline, which is known to be useful as an antidepressant and anorectic agent, and in the treatment of chemical dependencies, anxiety-related disorders, premature ejaculation, cancer and post-myocardial infarction.

These examples further yet show that the subject enzyme activity or polypeptide comprising the novel composition of matter is a monomer based on size exclusion data and gel migration data. Based on these data, the molecular weight of the subject polypeptide having the desired enzyme activity is from about 100,000 D to about 200,000 D and even more particularly from about 116,250 D to about 200,000 D and even more particularly about 158,000 D.

The present invention is illustrated by the following examples. The foregoing and following description of the present invention and the various embodiments are not intended to be limiting of the invention but rather are illustrative thereof. Hence, it will be understood that the invention is not limited to the specific details of these examples. For example, as those skilled in the art will appreciate based upon the present description and attendant claims, different purification schemes can be employed. Moreover, as described herein above, Hansenula polymorpha ATCC No. 26012, also means or includes Hansenula polymorpha ATCC No. 26012 deposited as ATCC No. 74449, and any other suitable mutant thereof.

The following medium, solutions and stocks are used in the examples provided below:

(a) 0.1 M KH₂PO₄, pH 7.0: 54.44 g into 3.9 L dH20, adjust to pH 7.0 with 20% KOH, final volume to 4.0 liters;

(b) Breaking Buffer to rupture the induced cells: 0.1 M KH₂PO₄, pH 7.0, 15% glycerol (measure volume and add 85% glycerol to a final concentration of 15%), 3 mM DTT (added immediately before use), Turkey Eggwhite Trypsin Inhibitor (1 mg/mL), and 2 mM EDTA;

(c) 2 mM stock of the compound of Formula (I), the substrate or inducer: Add 1.16 mL of a 5 mg/mL frozen solution of substrate (in ethanol) to 8.84 mL ethanol, bring to room temperature and mix to re-dissolve.

(d) 7.5 mM NADPH (prepared immediately before use): 12.4 mg NADPH into 2 mL 1% NaHCO₃;

(e) Packing Buffer: 10 mM KH₂PO₄, pH 7.0, 3 mM DTT (added immediately before use), and 2 mM EDTA;

(f) Binding Buffer/Desalting Buffer: 10 mM KH₂PO₄, pH 7.0, 3 mM DTT (added immediately before use), 2 mM EDTA, and 15% glycerol;

(g) Eluting Buffer for the affinity column: 10 mM KH₂PO₄, pH 7.0, 3 mM DTT (added immediately before use), 2 mM EDTA, 15% glycerol, and 5 mM NADPH.

A. Preparation of Hansenula polymorpha ATCC No. 26012 Crude Extract #1.

The growth medium (about 40 g/l of dextrose, about 10 g/l of nutrisoy flour, about 10 g/l of yeast extract, about 10 g/l of NaCl and about 10 g/l of K₂HPO₄, with the pH adjusted to about 7.0 with H₂SO₄), 50 mL in 250 mL flasks (41 flasks) was sterilized by autoclaving at 121° C. for 20 min.

Before inoculation, 4 mL of a Tween solution was then added to each flask (about 25 g of Tween® 80, about 100 g of glycerol and about 250 ml of distilled water, filter-sterilized).

About 250 microliters of a 17% frozen glycerol stock of Hansenula polymorpha ATCC No. 26012 was inoculated into all but one of the flasks (the uninoculated control). All of the flask cultures were then incubated at about 29° C., 40% relative humidity, with shaking at about 210 rpm. After about 23 hours, 1 mL of a stock solution (about 5 mg/ml in about 100% ethanol, final concentration of about 100 microgram/ml) of a racemic tetralone (compound of formula (I) comprising the compounds of formulae (IV) and (V), at about 5 mg/ml in ethanol) was added to each flask.

After about five days, the contents of the flasks that received Hansenula polymorpha ATCC No. 26012 were pooled into weighed 250 mL Nalgene® bottles (2.5 mL removed for an activity assay described below), spun for 15 min at 10,000 rpm in a Sorvall centrifuge, whereupon a wet cell weight was obtained (131.55 g total). Two grams of cells (wet weight) were resuspended per 10 mL of Breaking Buffer using a homogenizer (Janke & Kunkel IKA Labortechnik Ultra-Turrax T25 on the 8000 setting). The cells were broken in a Biospec Bead-Beater using 0.5 mm glass beads in the 365 mL chamber (up to 50% beads, or 183 mL) for 10 min (30 sec pulses with 1.5 min cooling periods, with air excluded from the chamber. Breakage of the cells was confirmed microscopically, the broken cells were spun for 15 min at 10,000 rpm to remove cell debris, 85% glycerol was added to the supernatant for a final concentration of 15% glycerol to stabilize the proteins, and the stabilized supernatant (482 mL) was frozen but for aliquots removed to ascertain the protein concentration and to assay for the presence of the desired enzyme activity.

The protein content of the crude extract was determined using a BioRad Protein Assay: the BSA (bovine serum albumin) samples were prepared as follows: 0.2 mg/mL (0.2 mL of a 5 mg/mL solution of BSA in 4.8 mL dH₂O ), 0.4 mg/mL (0.4 mL of a 5 mg/mL solution of BSA in 4.6 mL dH₂O), 0.6 mg/mL (0.6 mL of a 5 mg/mL solution of BSA in 4.4 mL dH₂O), and 0.8 mg/mL (0.8 mL of a 5 mg/mL solution of BSA in 4.2 mL dH₂O; the BioRad Dye Reagent was prepared by diluting 1 part Dye Reagent Concentrate with 4 parts milliQ dH₂O and filtering to remove particulates; 40 μl of each standard/sample was pipetted into the cuvette, 2 ml of diluted reagent was added to each cuvette and inverted to mix, and incubated at room temperature for 5 min (no longer than 1 hr.); and the absorbance was read at 595 nm using the Shimadzu UV160U spectrophotometer. The protein concentration of the crude extract was 6.58 mg/mL.

Three samples of about 2 mg each of the crude extract in separate test tubes were used to assay for the presence of the subject enzyme activity (the reductase) as follows: samples 1 and 2 each contained 2.4 mL of 0.1M KH₂PO₄ Buffer, pH 7.0, 0.1 mL of 7.5 mM NADPH (co-factor for the reductase), 0.3 mL of the crude extract, and 0.2 mL of 2.0 mM stock of the compound of Formula (I), the substrate, whereas sample 3 contained 2.5 mL of 0.1 M KH₂PO₄ Buffer, pH 7.0, 0.3 mL of the crude extract, and 0.2 mL of 2.0 mM stock of the substrate (i.e., no co-factor). All three samples were incubated at 29° C., 210 rpm, for 24 h.

The samples were the extracted by adding one ml of NaCl (sat'd.) to each tube culture. The contents of each tube culture (about 3 mL) was extracted with an equal volume of ethyl acetate (neat): the ethyl acetate was added, the tube culture was vortexed at motor speed 70, pulser 50 for 10 min each, spun for 20 min at 95% (about 2000 rpm) in an IEC centrifuge (IEC® Centrifuge, 300 Second Avenue, Needham Heights, Mass. 02194). The ethyl acetate phase (organic extract) was dried down, under nitrogen, in a water bath at 50° C. Each of the extracts, prepared as described above, was resuspended in about one ml of ethanol, and about 20 microliters of each of the resuspended extracts were separately analyzed by injection onto HPLC columns: Chiralcel OK guard columns (4.6×50 mm, Diacel Chemical Industries, LTD., 730 Springdale Drive, P.O. Box 564, Exton, Pa. 19341) coupled to a Chiralcel OK column (4.6×250 mm, Daicel). The compounds contained within each injected resuspended extract were separated isocratically at about 0.8 ml per minute in a mobile phase (ethanol:ethyl acetate, 85:15), and the compounds comprising the extracts were detected using a 996 PDA detector (Waters®, 34 Maple Street, Milford, Mass. 01757) set at 254 nm. Specific activity (reductase) was detected in samples 1 and 2 but not in sample 3, the sample that lacked the co-factor, where specific activity is calculated by dividing the percent amount of enantiomeric excess (% ee) of the (4S) tetralone produced by the amount of protein used (in mg): sample 1 (% ee of 12.7 divided by 2 mg of protein)=6.4, and sample 2 (% ee of 11.1 divided by 2 mg of protein)=5.6.

As is discernable from above, about 3 g of total protein containing the subject enzyme activity was generated by preparing this crude extract of Hansenula polymorpha ATCC No. 26012. This crude extract may be used as a positive (has the reductase activity) control.

B. Preparation of Hansenula polymorpha ATCC No. 26012 Crude Extract #2.

This crude extract was prepared substantially as provided in part A. of this example; however, the specific details are now provided.

The pooled cell broth was poured into two 1 L centrifuge bottles, and centrifuged at 7000 rpm for 20 min in a Sorvall RC-5B centrifuge, the supernatant was poured off, and the cells were harvested. The wet cell weight was 90 g. The cells were resuspended in Breaking Buffer (at 2 mg cells/10 mL Breaking Buffer, 90 g/450 mL). The Bead-Beater was filled with about 180 mL cold glass beads having 0.5 mm diameter, then filled to capacity with resuspended/homogenized cells, as provided above. The beating occurred in pulses: 30 sec on, 90 sec off, 6 times. The cells were examined for breakage. Two additional aliquots of equal volume of the remaining supernatant (given the volume limitations of the Bead-Beater) were processed as immediately described above, and the resultant extracts combined. The pooled extracts were then poured into 250 mL centrifuge bottles, and centrifuged at 10,000 rpm for 15 min in the Sorvall RC-5B centrifuge. The supernatants was poured off and combined to generate about 425 mL, to which 75 mL of glycerol stock was added, for protein stabilization. The total protein (determined as provided above) was 10.14 mg/mL, based on 500 mL total volume (425 mL supernatant+75 mL glycerol), or 5.07 g total protein. In addition, one mL retained from the pooled broth was assayed for the presence of the (4R) and (4S) alcohols which would be indicative of the presence of the subject enzyme activity as substantially provided above: to one mL of broth in a tube was added 0.5 mL sat'd. NaCl, 1 mL acetone (neat), and 1.5 mL ethyl acetate (neat); the tube was vortexed at motor speed 70, pulser 50 for 10 min each, spun for 20 min at 95% (about 2,000 rpm) in an IEC centrifuge (IEC® Centrifuge, 300 Second Avenue, Needham Heights, Mass. 02194). The ethyl acetate phase (organic extract) was dried down, under nitrogen, in a water bath at 50° C. Each of the extracts, prepared as described above, was resuspended in about 0.05 mL of acetonitrile for HPLC analysis as described above. The peak at 2.038 minute was the desired hydroxylated product (indicating the presence of the reductase), the non-reduced substrate showed at 2.756 min.

C. Ammonium Sulfate Precipitation of the Crude Extract #2.

The starting volume of the crude extract #2 was 410 mL to which 40 μl of a 10% polyethylenimine solution (Sigma lot: 97H04121-50% solution) per ml of crude enzyme, or 16.4 mL, was added. The tube was Inverted occasionally and maintained on ice. After 5 min, the mixture was centrifuged for 30 min at 12,000 rpm in the GSA rotor of the Sorvall centrifuge.

The supernatant was decanted. The volume was measured (410 mL). Solid ammonium sulfate (Sigma lot 39H5442, cat# A-4418), 0.28 g per ml of supernatant, was added with stirring to achieve 48% fractional saturation. Undissolved salt was filtered off (1.157 g), followed by centrifugation (12,000 rpm, 30 min, GSA rotor) of the eluent, with both the pellet and the supernatant being retained, and refrigerated overnight.

The volume of the supernatant was measured (450 mL). Solid ammonium sulfate, 0.18 g per ml of supernatant, was added with stirring to achieve 75% fractional saturation, followed by centrifugation (12,000 rpm, 45 min, GSA rotor), with both the pellet (#2) and the supernatant being retained Pellet (#2) was resuspended in 60 mL of Binding Buffer/Desalting Buffer, then desalted using an Amicon ultrafiltration cell concentrator (50 mL chamber) having a Millipore ultrafiltration membrane (YM10, 44.5 mm, lot K9EM K569). Through dialysis, the 60 mL was concentrated down to 20 mL, repeated 3×, and refrigerated overnight. The final volume was 26.5 mL. The concentrated preparation was assayed for total protein substantially as described above, with the concentrated preparation aliquot being diluted 1/40 in water (total protein concentration=30.03 mg/mL.

The reductase activity of the concentrated preparation was assayed as substantially described above, using crude extract #1 (6.58 mg/mL protein) as a positive control, and no protein as a negative control. The three samples in separate test tubes were used to assay for the presence of the subject enzyme activity (the reductase) as follows: sample 1 (crude extract #1) contained 1.16 mL of 0.1M KH₂PO₄ Buffer, pH 7.0, 0.025 mL of 7.5 mM NADPH (co-factor for the reductase), 0.15 mL of the crude extract, 0.12 mL of 125 mM MgCl₂, and 0.05 mL of 2.0 mM stock of the compound of Formula (I), the substrate; sample 2 (the concentrated prep from the ammonium sulfate precipitation) contained 1.27 mL of 0.1M KH₂PO₄ Buffer, pH 7.0, 0.025 mL of 7.5 mM NADPH (co-factor for the reductase), 0.03 mL of the concentrated prep, 0.12 mL of 125 mM MgCl₂, and 0.05 mL of 2.0 mM stock of the compound of Formula (I), the substrate; and sample 3 (no protein) contained 1.31 mL of 0.1M KH₂PO₄ Buffer, pH 7.0, 0.025 mL of 7.5 mM NADPH (co-factor for the reductase), 0.12 mL of 125 mM MgCl₂, and 0.05 mL of 2.0 mM stock of the compound of Formula (I), the substrate. The samples were each processed for HPLC analysis as provided in B. above. The specific activities were as follows: sample 1=9.3 (Units/mg), sample 2=16.5 U/mg, and sample=0.0 U/mg. The purification of the subject enzyme activity was 1.8 fold at this juncture, after the ammonium sulfate precipitation (16.5/9.3).

D. Blue Sepharose CL-6B Column Chromatography of the Preparation Prepared in C.

The column was purchased from Pharmacia Biotech: the column was equilibrated with 5 C.V. Binding Buffer in a fritted funnel, then 10 g of powder was packed (at about 10 mL/min offline using the Packing Buffer) into the 2.6 cm x about 9 cm column, with the mean bead size being about 90 microns (from about 45 to about 165 microns), the resuspended desalted prep from C. was loaded, and eluted by running at 5 mL/min flow, 2 C.V. of Binding Buffer equilibration at 0 NADPH, then the gradient of 0 to about 3 mM NADPH in Binding Buffer, in 10 C.V. (about 114.68 min total run time counting a 2 C.V. at 0 NADPH), with 5 mL fractions being collected, where the enzyme activity of interest eluted (Elution Buffer) in from about 0.8 to about 3 mM NADPH. The total protein in each fraction was determined substantially as provided above. The assay for reductase activity was performed as substantially described above on the pass through, and on fractions 31-139 (odd numbered). Specific activities (U/mg) based on the use of substantially the same protein per fraction (0.1 mg): positive control crude extract #1 (6.2 based on 0.99 mg protein), pass through (0.0) and selected fractions 31 through 139 as follows in Table 1 wherein F# means fraction number and (S.A.) means specific activity:

TABLE 1

| F# (S.A.) | F# (S.A.) | F# (S.A.) | F# (S.A.) | F# (S.A.) | F# (S.A.) |
|---|---|---|---|---|---|
| 31 (0.0) | 49 (20.5) | 67 (15.3) | 87 (10.2) | 107 (5.1) | 125 (0.00) |
| 33 (30.7) | 51 (20.5) | 69 (15.3) | 89 (10.2) | 109 (10.2) | 127 (0.00) |
| 35 (30.7) | 53 (20.5) | 71 (10.2) | 91 (10.2) | 111 (10.2) | 129 (0.00) |
| 37 (30.7) | 55 (15.3) | 73 (15.3) | 93 (5.1) | 113 (10.2) | 131 (0.00) |
| 39 (46.0) | 57 (15.3) | 77 (5.1) | 95 (10.2) | 115 (5.1) | 133 (0.00) |
| 41 (56.3) | 59 (20.5) | 79 (10.2) | 97 (10.2) | 117 (15.3) | 135 (0.00) |
| 43 (46.0) | 61 (15.3) | 81 (15.3) | 99 (10.2) | 119 (15.3) | 137 (0.00) |
| 45 (35.8) | 63 (15.3) | 83 (15.3) | 101 (10.2) | 121 (15.3) | 139 (0.0) |
| 47 (25.6) | 65 (15.3) | 85 (10.2) | 103 (10.2) | 123 (5.1) | — |

Based on these specific activities, fractions 33-121 were pooled (total volume of 410 mL), and then concentrated via an Amicon ultrafiltration cell concentrator (400 mLchamber) having a Diaflow ultrafiltration membrane (YM10, 76 mm, lot KOBN K151), to about 32 mL (3.47 mg total protein/mL).

E. Poros20HQ IV: Anion Exchange Column.

This column was purchased from PerSeptive Biosystems, Inc.: 4.6×100 mm (C.V.=about 1.662 mL), particle size 20 microns, the resuspended, desalted prep from D. above was loaded, with a 5 C.V. equilibration at starting conditions, run at 5 mL/min, gradient of Binding Buffer and 1 M NaCl (pH 8)(100:0 to 75:25)) over 40 C.V. (14.96 min total run time), with 2 mL fractions collected, where the enzyme activity of interest eluted at from about 0.08 M to about 0.24 M NaCl. The total protein in each fraction was determined substantially as provided above. The assay for reductase activity was performed as substantially described above on the pass through, and on fractions 4, 6, 16, 18, 20, 22, 24, 26, 28, 30, 34, 36, 38, 40.

Specific activities (U/mg) based on the use of substantially the same protein per fraction (0.05 mg): positive control crude extract #1 (3.1 based on 0.99 mg protein), pass through (0.0) and the fractions mentioned above as follows in Table 2:

TABLE 2

| F# (S.A.) | F# (S.A.) | F# (S.A.) | F# (S.A.) |
|---|---|---|---|
| 4 (0.0) | 20 (71.6) | 28 (40.9) | 38 (0.0) |
| 6 (0/0) | 22 (51.2) | 30 (30.7) | 40 (0.0) |
| 16 (51.2) | 24 (51.2) | 34 (20.5) | — |
| 18 (51.2) | 26 (51.2) | 36 (10.2) | — |

Based on the activity results, fractions 16-36 were pooled (total volume of 16.5 mL), desalted as substantially described above, and then concentrated via an Amicon ultrafiltration cell concentrator (400 mL chamber) having a Millipore Ultrafree Biomax-1OK membrane (lot: VSISO30) to about 6.4 mL (1.9096 mg total protein/mL).

F. DEAE Sepharose Column Chromatography.

This DEAE (diethylaminoethyl) Sepharose Fast Flow column was purchased from Pharmacia Biotech: 4.6×100 mm (column volume=about 1.662 mL), particle size of about 90 microns (from about 45 to about 165 microns), packed at about 2 mL/min with Binding Buffer and about 1 M NaCl (about pH 7)(100:0 to 50:50)) over 40 C.V. at about 1 mL/min (about 84.76 min total run time), and having a 5 C.V. equilibration at starting conditions (a 40 C.V. gradient, a 2 C.V. gradient to 0:100%, then a 4 C.V. “wash” of 0:100), loaded with substantially all of the pooled fractions of E. described above, with 2 mL fractions collected using the fraction collector, pass through being collected tubes 4-11, and sample elution starting at fraction 12, where the enzyme activity of interest eluted at from about 0.27 M to about 0.33 M NaCl. The total protein (mg/mL) in each fraction was determined substantially as provided above, and the results are shown below in Table 3:

TABLE 3

| F# (Protein) | F# (Protein) | F# (Protein) | F# (Protein) |
|---|---|---|---|
| 18 (0.0018) | 22 (0.0534) | 26 (0.8637) | 30 (0.2685) |
| 19 (0.0033) | 23 (0.1768) | 27 (0.8616) | 31 (0.1278) |
| 20 (0.006) | 24 (0.4427) | 28 (0.7563) | 32 (0.515) |
| 21 (0.0157) | 25 (0.7343) | 29 (0.5048) | 33 (0.0091) |

The assay for reductase activity was performed as substantially described above on fractions 18-33. Specific activities (U/mg) based on the use of substantially the same protein per fractions 21-32 (0.01 mg): positive control crude extract #1 (5.7 based on 0.99 mg protein), and the fractions mentioned above as follows in Table 4:

TABLE 4

| F# (S.A.) | F# (S.A.) | F# (S.A.) | F# (S.A.) |
|---|---|---|---|
| 18 (0.0) | 22 (51.2) | 26 (255.8) | 30 (51.2) |
| 19 (0.0) | 23 (204.6) | 27 (204.6) | 31 (51.2) |
| 20 (0.0) | 24 (255.8) | 28 (51.2) | 32 (51.2) |
| 21 (0.0) | 25 (255.8) | 29 (51.2) | 33 (222.4) |

Each of fractions 18-32 was evaluated using a NuPAGE gel to detemine the molecular weight of the polypeptide having the subject enzyme activity (the microbial reductase). The gel system was run according to the instruction booklet supplied with the gel kit, except that an antioxidant was not added to running buffer: (1) 10% Bis-Tris Gel with MES Running Buffer; (2) 20 microliter samples, with 1.5 microliters of Reducing Agent, and 3 microliters of 4× Sample Buffer; (3) 1 microliter Broad Range standard, 1.5 microliter Reducing Agent, 3 microliters 4× Sample Buffer, and 5 microliters H₂O. The gel was stained using the Hoefer Automated Gel Stainer program #5. As illustrated by Tables 3 and 4, most of the protein resided in fractions 23-29, which overlapped with most of the reducatse activity which resides in fractions 23-27. The NuPAGE staining, against the molecular weight standards, showed a band at a molecular weight of from about 116,250 D to about 200,000 D, the intensity of which appreciably correlated with the activity profile. Based on the activity results, fractions 18-32 were pooled (total volume of 16.5 mL), desalted as substantially described above, and then concentrated via an Amicon ultrafiltration cell concentrator (400 mL chamber) having a Millipore Ultrafree Biomax-1 OK membrane (lot: VSIS030) to about 1.0 mL. G. Superdex 200HR 10/30 (Size Exclusion Column).

This column was purchased from Pharmacia Biotech: 10×30 cm, bead diameter of from 13 microns to 15 microns, rehydrated with sodium phosphate buffer (comprising 10 mM KH₂PO₄, 3 mM DTT, 2 mM EDTA, 0.15 M NaCl, pH 6.8 for 1.5 C.V at a flow rate of 1 mL/min), with a total run time of 35.43 min.

The BioRad Gel Filtration Standard was evaluated under the following conditions (standard resuspended in 0.5 mLwater; 100 microliter injections): (1) 0.05 M NaH₂PO₄, 0.05 M NaHPO₄, 0.15 M NaCl, pH 6.8 (recommended mobile phase); (2) 10 mM KH₂PO₄, 3 mM DTT, 2 mM EDTA, pH 6.8; and (3) 10 mM KH₂PO₄, 3 mM DTT, 2 mM EDTA, 0.15 M NaCl, pH 6.8; with (3) providing the better resolution.

The desalted, concentrated, pooled fraction from F. described above, was loaded: total volume=1 mL; 10 injections of 100 microliters. Since each run looked very consistent, pooled all like fractions for tubes 11-21, 25, and 28/29 (both combined) and concentrated using Millipore Ultrafree Biomax-10K centrifugal unit (10,000 molecular weight cut off) (lot: VSIS030). Initial volume=10 mL except for 28/29: 20 mL. Centrifuged (Sorvall RC-5B centrifuge with SLA 600TC rotor) samples at 3500 rpm for 45 min (28/29 had two 50 min spins) resulting in a final volume of each fraction of about 0.75 mL.

The total protein (mg/mL) in each fraction was determined substantially as provided above in order to be able to use about the same protein from each fraction for the specific activity assessment.

The assay for reductase activity was performed as substantially described above on fractions 11-25, and 28/29. Specific activities (U/mg) based on the use of substantially the same protein per fractions 11-20 (0.01 mg): positive control crude extract #1 (3.1 based on 0.99 mg protein), and the fractions mentioned above as follows in Table 5:

TABLE 5

| F# (S.A.) | F# (S.A.) | F# (S.A.) | F# (S.A.) |
|---|---|---|---|
| 11 (307.0) | 15 (0.0) | 19 (51.2) | 28/29 (0.0) |
| 12 (255.8) | 16 (0.0) | 20 (51.2) | — |
| 13 (102.3) | 17 (0.0) | 21 (0.0) | — |
| 14 (51.2) | 18 (40.9) | 25 (0.0) | — |

Each of fractions 11-28/29 was also evaluated using a NuPAGE gel to detemine the molecular weight of the polypeptide having the subject enzyme activity (the microbial reductase). The gel system was run as described above in F.

Once again, consistent with the results shown in F. above, the reductase activity, most of which resides in fractions 11-14, and substantially in fraction 11, correlates with the degree of gel staining of a band (polypeptide) having a molecular weight of from about 116,250 D to about 200,000 D. Moreover, this particular polypeptide substantially shares a flow time through the size exclusion column with the bovine gamma globulin standard of 158,000 D.

Therefore, as those skilled in the art will appreciate, from the above illustrative examples as well as from the description and attendant claims, the present invention provides novel compositions of matter comprising an enzyme activity capable of stereoselectively reducing racemic tetralone to chiral tetralone. The specific activity of the enzyme activity (of the novel composition of matter derived from above described steps C. through H.) is particularly substantially higher than that of a crude extract used as a positive control for reductase activity.

It will be understood by those skilled in the art that advantages exist with respect to using a substantially purified enzyme rather than the intact microorganism, or a crude preparation therefrom such as, for example, less unwanted degradation due to proteases within the microorganism.

What is claimed is:

1. A composition of matter comprising an enzyme activity and having the following identifying characteristics:
(a) present in a fraction from a crude preparation of Hansenula polymorpha ATCC No. 26012, Hansenula polymorpha ATCC No. 74449, or any other suitable mutant thereof, where said crude fraction is prepared by: inducing cells of Hansenula polymorpha ATCC No. 26012, Hansenula polymorpha ATCC Hansenula polymorpha ATCC No. 74449, or any other suitable mutant thereof, with an amount of the compound of Formula (I) under suitable conditions to permit the induction of the enzyme activity capable of stereoselectively reducing racemic tetralone to chiral tetralone, centrifuging said induced cells, resuspending said centrifuged cells in a breaking buffer comprising beads, rupturing said resuspended cells under suitable conditions to permit disruption of said cells and retention of an appreciable amount of said enzyme activity, centrifuging said breaking buffer after said rupturing, retaining the supernatant of said centrifuged breaking buffer, and adding a protein stabilizing agent to said supernatant;
(b) present in a fraction of the fraction described in (a) above, where said fraction is obtained by: adding a DNA precipitating agent to an amount of said fraction described in (a) above, centrifuging said crude extract, retaining the supernatant of said centrifuged crude extract, adding a protein precipitating agent to said supernatant of said centrifuged crude extract to achieve about 48% fractional saturation, centrifuging said supernatant having about 48% fractional saturation, retaining said supernatant and adding a protein precipitating agent to said supernatant to achieve about 75% fractional saturation, centrifuging said supernatant having about 75% fractional saturation, retaining the pellet resulting from said centrifugation, resuspending the proteins comprising said pellet in a buffer and desalting and then concentrating said proteins in said buffer;
(c) present in a fraction of the fraction described in (b) above, where said fraction is obtained by: loading an amount of said fraction described in (b) above onto a column comprising a material capable of reversibly associating with said proteins comprising said fraction described in (b) and having said enzyme activity, eluting said reversibly associated proteins from said column using an NADPH gradient, assaying each eluted fraction for said enzyme activity, and pooling said eluted fractions having said enzyme activity;
(d) present in a fraction of the fraction described in (c) above, where said fraction is obtained by: desalting an amount of said fraction described in (c) above, loading an amount of said desalted fraction onto a column comprising an anion exchange material and capable of reversibly binding said proteins of said desalted fraction and having said enzyme activity, eluting said reversibly associated proteins from said column using a salt gradient, assaying each eluted fraction for said enzyme activity, and pooling said eluted fractions having said enzyme activity;
(e) present in a fraction of the fraction described in (d) above, where said fraction is obtained by: desalting an amount of said fraction described in (d) above, loading an amount of said fraction described in (d) above onto a column comprising a weak anion exchange material and capable of reversibly binding said proteins of said desalted fraction and having said enzyme activity, eluting said reversibly associated proteins from said column using a salt gradient, assaying each eluted fraction for said enzyme activity, and pooling said eluted fractions having said enzyme activity; and
(f) present in a fraction of the fraction described in (e) above, where said fraction is obtained by: desalting an amount of said fraction described in (e) above, concentrating said desalted fraction, loading an amount of said concentrated fraction onto a column comprising a size exclusion material, and eluting a fraction comprising a polypeptide of from about 110,000 D to about 200,000 D and having said enzyme activity;
where said activity is present in said fractions when an amount of racemic tetralone is stereoselectively reduced to an amount of chiral tetralone.

2. A process for the stereoselective reduction of a compound of Formula (i) to compounds of Formulae (II) and (III) 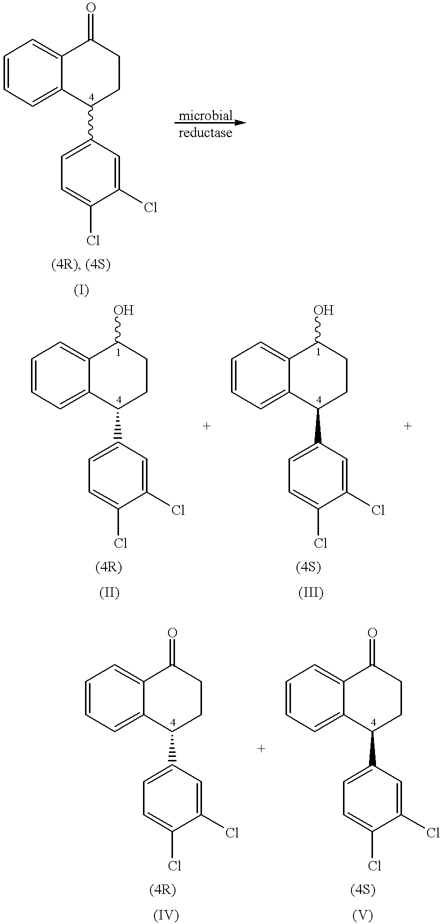
which comprises: contacting a compound of Formula (I) with a composition of matter comprising an enzyme activity capable of accomplishing the subject reduction and a co-factor for said enzyme, and incubating the resulting mixture under conditions sufficient to yield more of the compound of Formula (II) than the compound of Formula (III), thus leaving more of the compound of Formula (V) unreacted than the compound of Formula (IV) unreacted; where said composition of matter is as defined in claim 1.
